(19) United States
(12) Patent Application Publication
(10) Pub. No.: US 2012/0021452 A1
(43) Pub. Date: Jan. 26, 2012

(54) Belt Agitation System For Culture Bottles

(75) Inventor: James C. Bishop, Columbia, MO (US)

(73) Assignee: BIOMERIEUX, INC., Durham, NC (US)

(21) Appl. No.: 13/188,233

(22) Filed: Jul. 21, 2011

Related U.S. Application Data
(60) Provisional application No. 61/400,201, filed on Jul. 22, 2010.

(51) Int. Cl. C12Q 1/02; C12M 1/02; C12N 1/20; C12M 1/34

(52) U.S. Cl. 435/29; 435/288.7; 435/289.1; 435/252.1

(57) ABSTRACT

A specimen culture container agitation assembly includes an endless belt having a multitude of container-receiving clips attached thereto, each of which is adapted for retaining a specimen culture container to the belt. The belt is oriented vertically. The assembly further includes upper and lower pulleys over which the belt is driven. A drive motor drives the belt around the pulleys. Agitation of the specimen culture container is achieved by rotation of the belt over the pulleys and the retaining of the specimen culture container by the clip.

### CROSS REFERENCE TO RELATED APPLICATION

This application claims the benefit of U.S. Provisional Patent Application No. 61/400,201, entitled “Belt Agitation System for Culture Bottles”, filed Jul. 22, 2010, which is incorporated herein.

### FIELD OF THE INVENTION

This invention relates to a method and system for agitating culture specimen containers such as for example culture containers placed in an automated bacterial growth detection instrument. Specimens are incubated and agitated at a controlled temperature to promote bacterial growth within the specimen container.

### BACKGROUND OF THE INVENTION

Rapid and accurate processing of hospital patient samples is critical for diagnosing the illness and administering the correct bacteria-destroying drug. Instruments currently exist on the market that detect the growth of a microorganism in a biological sample. One such instrument is the BacT/ALERT® 3D instrument of the present assignee bioMérieux, Inc. The instrument receives a culture bottle containing a test sample, e.g., from a human patient. The instrument incubates the bottle and periodically during incubation an optical detection unit in the incubator analyzes a colorimetric sensor incorporated into the bottle to detect whether microbial growth has occurred within the bottle. The optical detection unit, bottles and sensors are described in the patent literature, see U.S. Pat. Nos. 4,945,060; 5,094,955; 5,162,229; 5,164,796; 5,217,876; 5,795,773; and 5,856,175, the entire content of each of which is incorporated by reference herein. Other prior art of interest relating generally to the detection of microorganisms in a biological sample includes the following patents: U.S. Pat. No. 5,770,394, U.S. Pat. No. 5,518,923; U.S. Pat. No. 5,498,543, U.S. Pat. No. 5,432,061, U.S. Pat. No. 5,371,016, U.S. Pat. No. 5,397,709, U.S. Pat. No. 5,344,417, U.S. Pat. No. 5,374,264, U.S. Pat. No. 6,709,857; and U.S. Pat. No. 7,211,430.

Existing culture detection systems process bottle samples inserted into racks. Mounted to the back of each rack is a circuit board containing optical detectors for each bottle. Multiple detectors require a simple and inexpensive design. Optical detection designs optimized for rapid detection would be too costly for individual bottle location.

In current practice, rack assemblies either mount into drawers or stack vertically behind a door. The drawers minimize instrument desk space by orienting the racks lengthwise to the pull direction. Opening the drawers exposes the samples to ambient air temperature which can slow bacterial growth and increase the detection time. Arranging the racks behind a door improves temperature control but typically results in a significantly larger instrument.

### SUMMARY OF THE INVENTION

In a first aspect, a specimen culture container agitation assembly is provided including an endless belt having a multitude of container-receiving clips attached thereto. Each of the clips is adapted for receiving and retaining a specimen culture container (e.g., a culture bottle). The belt is oriented vertically (i.e., in a vertical attitude). The assembly includes an upper pulley and a lower pulley over which the belt is driven. A drive motor drives the belt over the pulleys. Agitation of the specimen culture container is achieved by rotation of the belt over the upper and lower pulleys and the retaining of the specimen culture container by the clip.

In one possible configuration, the assembly includes an optical scanner or detection unit fixed in position proximate to the belt. The motor operates to move the endless belt and specimen containers attached thereto to a reading position adjacent to the optical scanner. The optical scanner or detection unit thereby serves to provide a detection feature for a plurality of specimen containers (e.g., for detecting microbial growth in each of the containers).

In another aspect, a detection instrument (e.g., a culture instrument or blood culture instrument) is disclosed for analyzing specimen containers for microbial growth. The instrument includes a multitude of the agitation assemblies in the form of the vertically-oriented endless belt, upper and lower pulleys and container receiving and retaining clips. The agitation assemblies are vertically oriented and spaced from each other in a radial fashion to save space and provide a compact arrangement for agitation of the containers. Each of the agitation assemblies includes at least one optical scanner or detection unit for interrogation of specimen containers held in the respective agitation assembly. In one possible configuration, the multitude of agitation assemblies are mounted to an indexing mechanism or carousel for rotation of the agitation assemblies about a vertical axis. The instrument may be configured to operate entirely robotically, and to that end may include a robotic manipulator or robotic arm for gripping the specimen containers at a loading position and automatically inserting the specimen containers into the retaining clips. In yet another aspect, the detection instrument may include a climate-controlled interior chamber (or incubation chamber), for maintaining an environment to promote and/or enhance growth of any microorganisms that may be present in the specimen container.

In still another aspect, a method of agitating at least one specimen container or bottle is disclosed, comprising the steps of: providing a vertical endless belt with a multitude of retaining clips each of which securely retain a specimen container therein; and rotating the endless belt and thereby the specimen containers in a vertical manner over upper and lower pulleys. The container agitation is accomplished when the containers rotate around the pulleys. Because the containers do not slip within the clip retention assemblies the containers are essentially inverted (about the horizontal axis) as they rotate around the pulleys.

The method may include optional features including a step of incubating the specimen container while performing the agitation of the specimen container.

As another example, movement of the belt continues without interruption as a specimen container held therein is moved past a fixed optical scanner (detection unit) interrogating a sensor incorporated within the specimen container. A single fixed optical scanner for all the bottles held in a belt agitation system simplifies the design of the instrument, and allows for continuous scanning of the specimen containers. Alternatively, the belt could be temporarily stopped so as to position a specimen container held therein proximate to the optical scanner interrogating the sensor incorporated within the specimen container.

### BRIEF DESCRIPTION OF THE DRAWINGS

FIG. 1 is an isometric view of a culture detection instrument which includes a belt agitation system placed behind the cover panels and illustrated in the subsequent figures. The upper front cover of the instrument has a monitor for viewing instrument status and an access door for bottle loading. Below the front door is a negative bottle waste drawer.

FIG. 2 is an isometric view of the instrument of FIG. 1 outside covers removed to show the agitation module assemblies in a radial configuration in order to minimize space and maximize bottle density within the instrument.

FIG. 3 is a top view of the agitation module assemblies of FIG. 2 showing the radial orientation.

FIG. 4 is an isometric view of the culture detection instrument of FIG. 1 with the front door opened the show a loading system and robot manipulator arm for inserting and removing the bottles into the agitation module assembly.

FIGS. 5A and 5B are isometric views of one of the agitation module assemblies of FIGS. 2-4, showing the Agitation Belt Assembly, Agitation Belt Drive Motor, and Bottle Sensor Optical Scanner. Bottles rotate past the fixed Optical Scanner and are read only at that location, thereby eliminating the need for multiple optical detection systems, one for each bottle as in prior art systems.

FIG. 6 is an isometric view of the Agitation Belt shown isolated from the rest of the structure in the agitation module assembly, showing the equal spaced profile pads which provide for mounting bottle retention clips which hold the bottles to the belt.

FIG. 7 is an isometric view of the bottle retention clip which attaches to the profile pads of FIG. 6, shown holding one of the bottles. The retention clip is split to allow the sides to be spring loaded for bottle retention. The split also provides an opening for a simple bottle removal mechanism.

FIG. 8 is an isometric view of an injection molded plastic bottle retention clip of FIG. 7 but without the bottle. A radial profile feature is formed in the sides of the clip near the end of the clip for engaging into a corresponding groove or detent formed in the bottle, which is shown in FIG. 7. The spring-loaded sides and rear retention profile securely retain and accurately locate the bottle position as it rotates past the optical scanner of FIGS. 5A and 5B.

### DETAILED DESCRIPTION

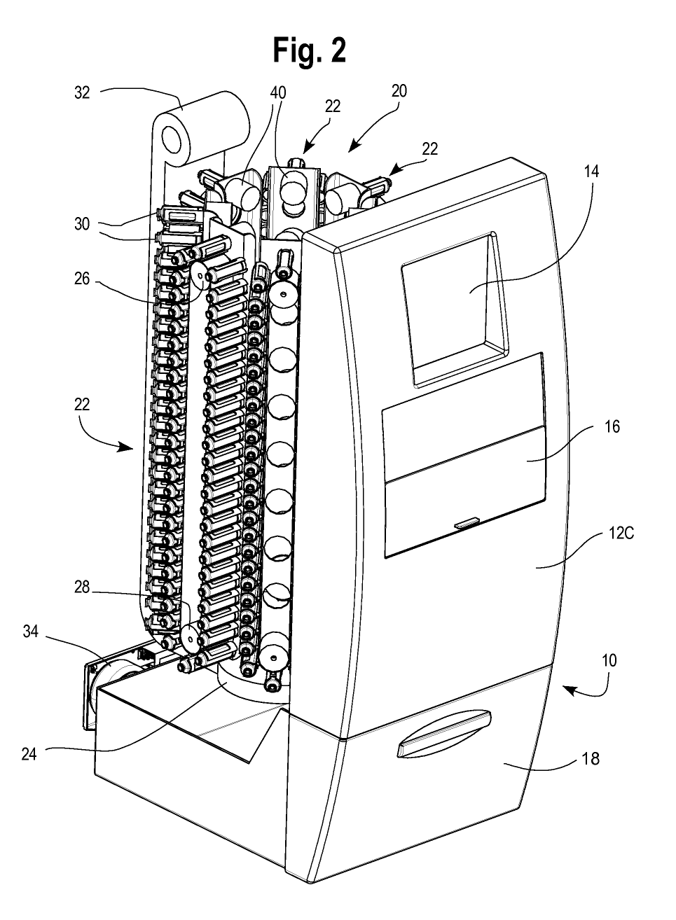

A specimen container agitation assembly and a method of agitating at least one specimen container are described herein, wherein the agitation assembly comprises an endless belt having a multitude of container-receiving clips attached thereto. The disclosure also describes a detection instrument (e.g., a culture instrument or blood culture instrument) for analyzing specimen containers for microbial growth, the instrument comprising at least one specimen container agitation assembly. The specimen container 30 (shown for example in FIGS. 2-5 and 7) described herein is in the form of a standard culture bottle. However, as one of skill in the art would readily appreciate, other design configurations are possible and are well known in the art.

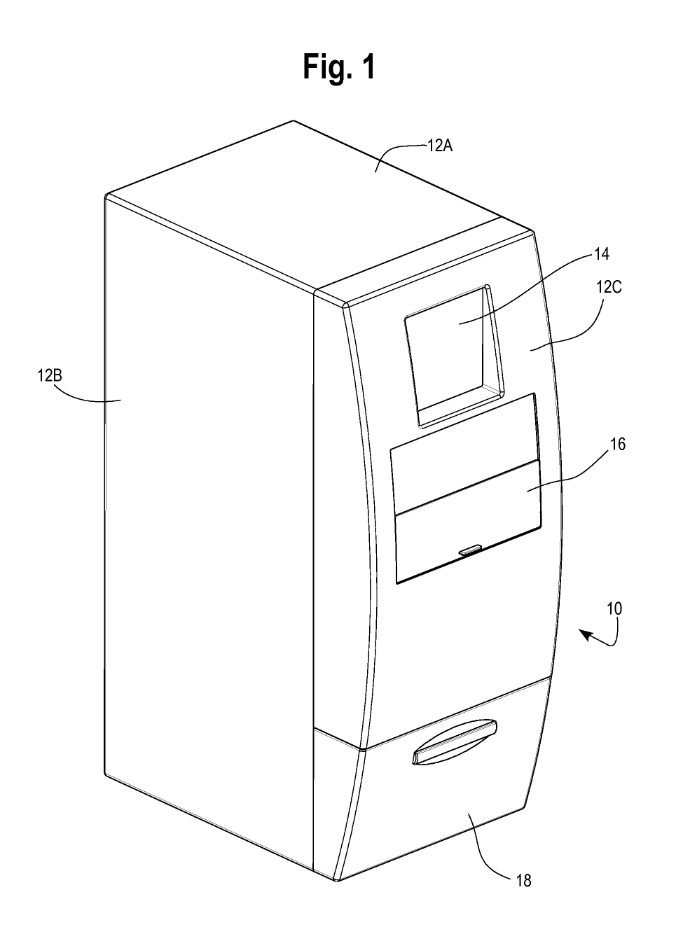

FIG. 1 is an isometric view of a culture detection instrument 10 which includes a belt agitation system placed behind cover panels enclosing the agitation system (illustrated in the subsequent figures as item 20). As shown, the culture detection instrument comprises left and right side panels 12A, top and bottom panels 12B and front and back panels 12C. As is well known in the art, the detection system 10 may include a climate-controlled interior chamber (or incubation chamber), as defined by the enclosed created by the side panels 12A, top and bottom panels 12B and the front and back panels 12C. The enclosed chamber may include a heating element or other features for maintaining an environment to promote and/or enhance growth of any microorganisms that may be present in the specimen container 30.

The upper front cover 12C of the instrument is in the form of a door and has a monitor 14, for viewing instrument status or for providing an operator or laboratory technician with status information regarding containers loaded into the detection system. As shown, the upper front cover 12C also includes an access door 16 for specimen container (e.g., a culture bottle) loading or unloading (e.g., of containers determined to be positive for microorganism growth). Below the access door 16 is a negative bottle waste drawer 18, for containers that tested negative for the presence of microorganism growth. As is well known in the art, specimen containers or bottles 30 in which a microbial agent is present are termed “positive” herein. Whereas, specimen containers or bottles 30 determined as negative for microbial growth after a designated time has passed are referred to herein as a “negative” containers or bottles.

In an overall sense, the instrument 10 functions to receive specimen containers or bottles 30 (FIGS. 2-4) containing a sample to be tested, incubates the containers 30, performs agitation on the containers during the incubation, and periodically interrogates a sensor formed in the container to determine whether microbial growth has occurred within the specimen container. The incubation and interrogation aspects are not particularly important to the instant agitation invention and are described only briefly.

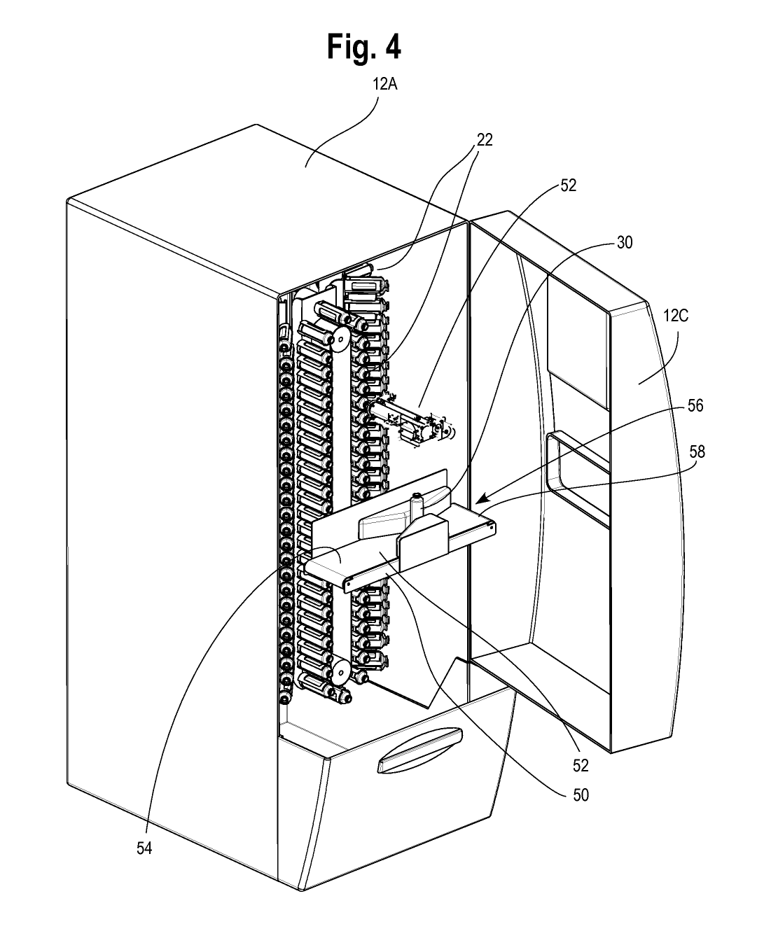

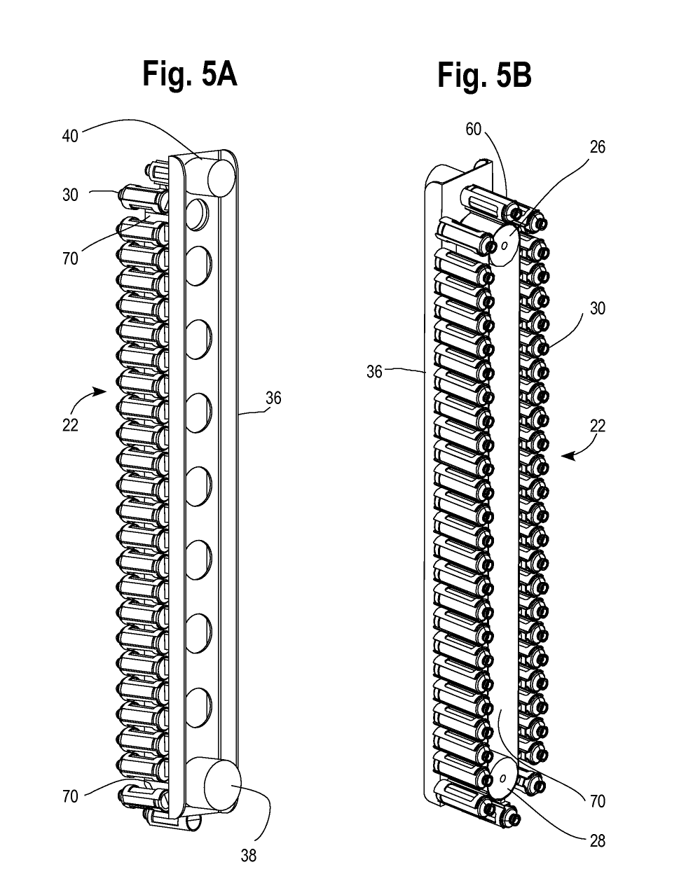

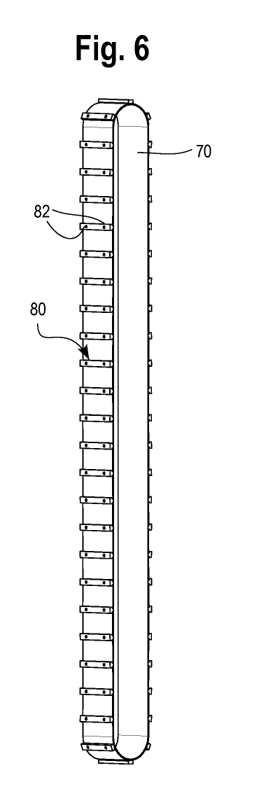

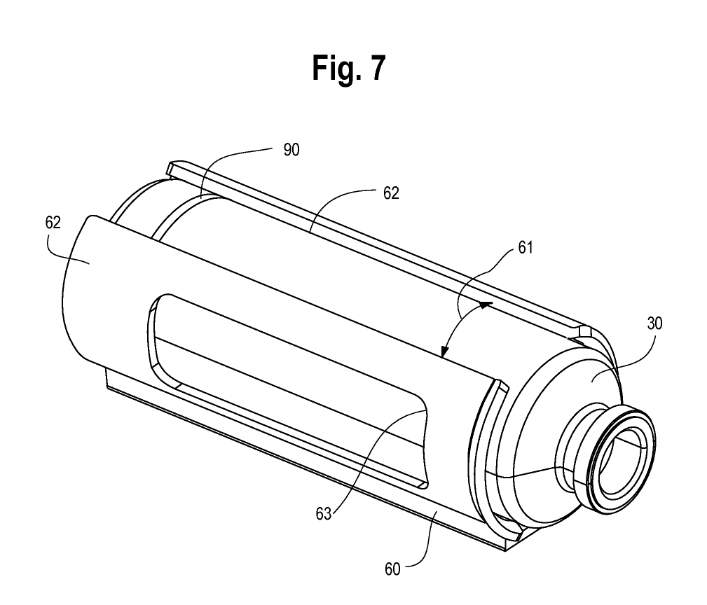

FIG. 2 is an isometric view of the instrument 10 of FIG. 1 with the side covers 12A, top cover 12B and back cover removed to show the agitation system 20 of this disclosure. The agitation system 20 takes the form of a number of identical agitation module assemblies 22 in a radial configuration in order to minimize space and maximize container or bottle density within the instrument. The agitation module assemblies 22 are mounted to a rotatable turntable or carousel 24 which functions as an indexing mechanism. The turntable 24 rotates about a vertical axis so as to selectively place any of the assemblies 22 opposite to a container loading position 56 (FIG. 4) located behind the access door 16. Each agitation module assembly 22 is mounted vertically and equal spaced around the turntable 24. The agitation module assemblies 22 include an upper idler pulley 26 and a lower drive belt pulley 28 over which a vertically-oriented endless belt 70 (FIGS. 5B, 6) is fitted. The pulleys 26 and 28 are gear toothed pulleys. The drive belt pulley 28 is attached to an electric motor 38 (FIG. 5A). The upper idler pulley 26 is attached to a belt tensioning assembly (not shown). Specimen containers (bottles) 30 are retained in individual container receiving and retention clips 60 (FIG. 7) which are fastened to the belt 70 via profile pads 80 (FIG. 6). The belt 70 is rotated by means of a drive motor 38 (FIG. 5A) thereby carrying the bottles around the path of the belt. The bottle agitation is accomplished when the bottles 30 rotate around the endless belt, the top idler and drive belt pulleys. Because the bottles do not slip within the clips 60 the bottles 30 are essentially inverted (about the horizontal axis) as they rotate around the pulleys 26 and 28. There may be a short dwell time between top and bottom bottle 30 agitation depending on the distance between the top and bottom pulleys as well as the belt rotation rate.

When each bottle 30 reaches the top position of the belt 70 immediately above the upper idler belt pulley 26, the belt 70 continues to move as the bottles 30 are read by an optical scanner or detection unit 40 (FIGS. 2, 3, 5A). The optical scanner or detection unit 40 includes suitable optical or detection arrangements for interrogating a sensor incorporated into the bottle 30, as is well known in the art. For example, the optical scanner 40 could have the light source and detector arrangements described in the patent literature cited earlier in this document, and commercialized in the culture detection instruments such as the BacT/ALERT® 3D instrument of the applicant's assignee bioMerieux, Inc. In one embodiment, the scanner or detection unit 40 is optimized for detecting bottle sensor color changes (e.g., a colorimetric sensor). The optical scanner or detection unit is positioned on the radius of rotation of the bottle 30 retention clips 60, thereby centering the scanner 40 with respect to the center of the base of the bottle 30 as held by the retention clip. The belt 70 is moved to the next position and the adjacent bottle 30 is moved past the scanner or detection unit 40. Alternatively, the belt 70 can be stopped briefly for reading of a specimen container by the scanner or detection unit 40.

FIG. 2 also shows a blower/heater system 32 which provides warm air to the enclosure defined by the cover panels (i.e., the incubation chamber) of FIG. 1 thereby establishing an incubation environment for the specimen containers during agitation and reading. A power supply 34 provides power to the motors for the agitation system 20, detection units and other electronic components of the instrument 10. These details are not particularly important and therefore a further description is omitted.

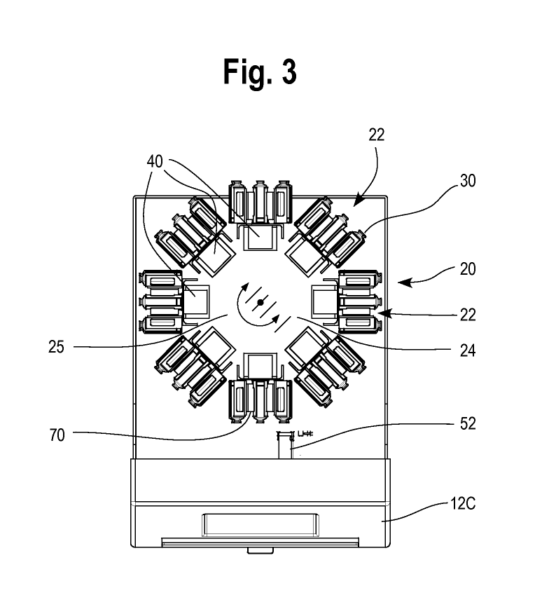

FIG. 3 is a top view of the agitation module assemblies of FIG. 2 showing the radial orientation of the agitation module assemblies 22. The rotatable turntable or carousel 24 rotates about the vertical axis (e.g., either clock-wise or counter clock-wise) as indicated by the arrow 25. As on of skill would readily appreciate, rotation of the turntable or carousel 24 allows for agitation of the specimen container 30. In another embodiment, the turntable or carousel 24 can be rotated so as to bring any of the bottle positions (e.g., bottle 30 located along the right hand side of the belt 70) into a position opposite a robot manipulator or robotic arm 52 so as to enable a bottle 30 to be inserted into one of the receiving and retaining clips 60 in the belt 70, or to remove one of the bottles 30 from the receiving and retaining clip 60. To get a given bottle 30 to the correct height corresponding to the height of the robot manipulator 52, the belt 70 may need to be rotated some amount around the upper and lower pulleys 26 and 28 so as to place the clip 60 holding the bottle of interest to the right side of the belt 70 at the proper height and in position for insertion or extraction of the bottle.

FIG. 4 is an isometric view of the culture detection instrument 10 of FIG. 1 with the front door 12C opened showing a loading and unloading system 50 and robot manipulator arm 52 for inserting and removing the containers 30 into the agitation module assembly 22. The loading and unloading system 50 in this configuration includes a conveyor belt 54 which advances bottles 30 to a holding position or loading position 56 proximate to the robot manipulator 52. In general, any type of robotic manipulator 52 known in the art can be used. The illustrated robot manipulator 52 shown in FIG. 4 is rotatable about a horizontal axis and includes a gripping structure or fingers which translates back and forth. The robot manipulator 52 rotates to a position directly above the holding position where the bottle 30 is shown located in FIG. 4. The gripping structure extends to grip the bottle 30. The robot manipulator then rotates about the horizontal axis to the position shown in FIG. 4 and the gripping structure extends to insert the bottle 30 into the clip 60 directly opposite from the robot manipulator 52. The gripping structure is then released and the bottle held in position by the belt retaining clip 60 of FIG. 7. As one of skill in the art would readily appreciate, the loading and unloading system 50 and robotic manipulator 52 can be supported using one or more support rods or other similar support structure within the instrument 10 (support rods have been omitted from the figures for clarity). For example, in one embodiment, the robotic manipulator 52 may be supported by a horizontally orientated support rod within the instrument. In accordance with this embodiment, the robotic manipulator 52 may move along the support rod (e.g., from right to left, or vice versa, in a horizontal manner) and/or the robotic manipulator 52 or support rod itself may be rotatable about the horizontal axis, thereby allowing the robotic manipulator 52 to change the orientation of the container or bottle 30 from a vertical orientation (e.g., an up-right orientation) to a horizontal orientation (i.e., such that the bottle 30 is laying in it's side). In another embodiment, the robotic manipulator 52 may be supported by a vertical support rod, thereby allowing the robotic manipulator 52 to traverse up and down the support rod (i.e., along a vertical axis). Other features of the loading system 50 and robotic manipulator 52 of FIG. 4 may include features of the automated loading and bottle gripping features described in: (1) pending U.S. application Ser. No. 12/800,467 filed on May 14, 2010, entitled “Combined detection instrument for culture specimen containers and instrument for identification and/or characterization of a microbial agent in a sample”; and/or (2) U.S. application Ser. No. 12/780,126 filed on May 14, 2010, entitled “Automated microbial detection apparatus; the contents of which are incorporated by reference herein.

The robotic manipulator 52 is also used to unload positive and negative bottles 30. In the case of a positive bottle 30, the robotic manipulator 52 will retrieve the bottle from the retaining clip 60 and move the bottle to the loading and unloading system 50. Once there the gripping mechanism will release the bottle 30 and optionally the conveyor can move the bottle to a access station 58, where a user or laboratory technician can retrieve the bottle. In a similar manner, the robotic manipulator 52 can retrieve a negative bottle 50 from the retention clip 60 a deposit the bottle into a waste container located behind the negative bottle waste drawer 18 (FIG. 1).

FIGS. 5A and 5B are isometric views of one of the agitation module assemblies of FIGS. 2-4 with FIG. 5A showing the assembly mostly from the rear and FIG. 5B showing the assembly from the front. FIG. 5A shows the agitation belt 70, agitation belt drive motor 38, and the optical scanner or detection unit 40. FIG. 5B shows the upper idler pulley 26 and a lower drive belt pulley 28. Bottles 30 rotates past the fixed optical scanner or detection unit 40 and are read only at that location, thereby eliminating the need for multiple optical detection systems, one for each bottle as in prior art systems. An aluminum channel 36 provides a rigid mounting base for assembly 22.

FIG. 6 is an isometric view of the agitation belt 70 shown isolated from the rest of the structure in the agitation module assembly 22, showing equal-spaced profile pads 80 which provide a structure for mounting the bottle receiving and retention clips of FIG. 7 which hold the bottles 30 to the belt.

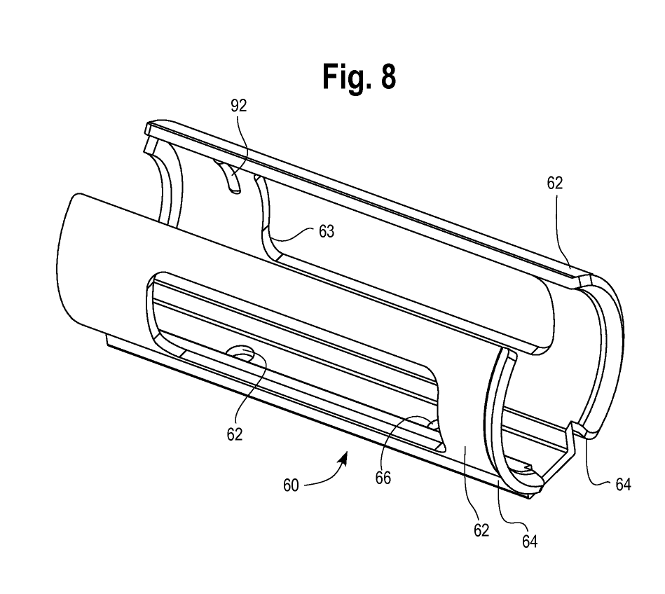

FIG. 7 is an isometric view of the bottle receiving and retention clip 60 which attaches to the profile pads of FIG. 6, shown holding one of the bottles 30. The retention clip 60 is split (61) to allow the sides 62 to be spring-loaded for bottle retention. The split 61 (gap between the sides 62) also provides an opening for a simple bottle 30 removal mechanism. The clip 60 is shown in isolation in FIG. 8. The clip includes a pair of channels 64 which are complimentary to the profile of the pads 80 of FIG. 6 allowing the clip 60 to be slid over the pads 80. The clips 60 are secured to the pads 80 by means of a threaded fastener (not shown) that extends through the holes 66 and is received in threaded holes 82 in the pads 80 (FIG. 6). The retention clips 60 could be fastened to the belt in any convenient fashion and the details of FIGS. 6-8 are not considered critical.

The split 61, as well as the side slots 63 also provides openings for a bar code scanner (not shown) to read bar codes on the fly during agitation. The agitation belt drive motor has an integrated encoder (not shown) and would always know the location of each bottle mounting pad 80. Each specimen container, identified by its bar code, would have a specific belt location. If the identified bottle 30 was mistakenly moved to another clip location, the instrument would identify the misplaced bottle during bar code scanning and trip an “anomalous bottle loading” flag or the like.

In one possible configuration, the bottle 30 includes a detent ring or groove 90 (FIG. 7) which extends around the circumference of the side wall of the bottle 30. The clip 60 includes a bottle retention profile 92 in the form of a raised ridge which snaps into the groove 90 when the bottle 30 is fully inserted into the clip 60 to thereby securely grip the bottle and insure that the bottle is in the correct or “home” position for reading and not displaced away from the scanner 40, which could cause improper readings of the sensor included in the bottle 30. The clips 60 can be made using plastic injection molding.

The clips 60 are split as shown in FIGS. 7 and 9 with an internal diameter slightly smaller than the bottle 30 outer diameter. The sides 62 provide some resiliency to expand and grip the bottle 30. As the bottle 30 enters the retention clip 60, the sides 62 expand applying a uniform force around the bottle surface. The bottle is locked into position as the engagement groove 90 near the end of the bottle snaps into the corresponding profile 92 on the inside surface of the sides 62. The split in the retention clip 60 also provides an opening for a simple mechanical extraction arm to remove negative bottles 30 into a waste container, by means of grasping the head of the container and rotating the bottle 30 out of the clip by forcing the sides 62 to expand and free the bottle.

While the embodiment of FIG. 3 features robotic loading and unloading, in alternative configurations the bottles 30 could be accessed manually.

From the foregoing description, it will be appreciated that a belt agitation assembly has been described for holding a multitude of specimen containers which provides for agitation of specimen containers, maximizes bottle 30 density (see arrangement of FIG. 3), minimizing instrument size, and simplify optical detection of a sensor incorporated within the specimen containers by only requiring one optical detector/scanner (40) per belt. Of course, two or more detectors/scanners 40 could be included in an instrument but it is generally not necessary.

The appended claims are considered further descriptive of the disclosed inventions.

I claim:

1. A specimen culture container agitation assembly comprising:
an endless belt having a multitude of container-receiving clips attached thereto, each of which is adapted for receive and retaining a specimen culture container to the belt, the belt oriented in a vertical attitude;
an upper pulley and a lower pulley over which the belt is driven; and
a drive motor driving the belt wherein agitation of the specimen culture container is achieved by rotation of the belt over the upper and lower pulleys and the retaining of the specimen culture container by the clip.

2. The agitation assembly of claim 1, wherein said specimen culture container is a culture bottle.

3. The agitation assembly of claim 1, further comprising an optical detection unit positioned proximate to the belt wherein the motor operates to move the endless belt and specimen containers attached thereto to a reading position adjacent to the optical detection unit, the optical detection unit thereby serving to provide a feature for detecting microbial growth in said containers.

4. The agitation assembly of claim 1, wherein the clip includes first and second opposite sides separated by a split.

5. The agitation assembly of claim 1, further comprising a multitude of pads placed along the belt, the clips attached to the pads.

6. The agitation assembly of claim 4, wherein the clips further comprise a container retention feature for engaging with a corresponding feature on the exterior of the specimen container.

7. A detection instrument for analyzing specimen containers for microbial growth, comprising:
a multitude of the agitation assemblies of claim 1, the agitation assemblies vertically oriented and spaced apart from each other in a radial fashion;
each of the agitation assemblies including at least one optical scanner for interrogation of specimen containers held in the respective agitation assembly.

8. The detection instrument of claim 7, wherein the multitude of agitation assemblies are mounted to an indexing mechanism rotating the agitation assemblies about a vertical axis.

9. The detection instrument of claim 7, further comprising a robotic manipulator gripping the specimen containers and inserting the specimen containers into the retaining clips.

10. A method of agitating a specimen container, comprising the steps of:
providing a vertical endless belt with a multitude of retaining clips each of which securely retain a specimen container therein; and
rotating the endless belt and thereby the specimen containers in a vertical manner over upper and lower pulleys.

11. The method of claim 10, wherein said specimen culture container is a culture bottle.

12. The method of claim 10, further comprising the step of incubating the specimen container while performing the agitation of the specimen container.

13. The method of claim 10, further comprising the step of stopping movement of the belt so as to position a specimen container held therein proximate to an optical detection unit interrogating a sensor incorporated within the specimen container.

14. The method of claim 13, wherein the optical detection unit is located proximate to one of the upper and lower pulleys.

15. The method of claim 10, further comprising continuously moving the belt while a specimen container held therein is moved past and proximate to an optical detection unit interrogating a sensor incorporated within the specimen container.
